(19) United States
(12) Patent Application Publication
(10) Pub. No.: US 2003/0086886 A1
(43) Pub. Date: May 8, 2003

(54) Hair styling composition containing particles

(76) Inventor: Sanjeev Midha, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/264,236

(22) Filed: Oct. 3, 2002

Related U.S. Application Data
(60) Provisional application No. 60/326,681, filed on Oct. 3, 2001.
(60) Provisional application No. 60/328,179, filed on Oct. 10, 2001.

(51) Int. Cl. A61K 7/06; A61K 7/09

(52) U.S. Cl. 424/70.2; 424/70.1

(57) ABSTRACT

The compositions of the present invention relate to improved hair styling compositions having from about 0.1% to about 15%, by weight, of an adhesive polymer having a weight average molecular weight of greater than about 20,000; from about 0.01% to about 20%, by weight, of particles; and an aqueous carrier.

### CROSS REFERENCE TO RELATED APPLICATION

The application claims the benefit of U.S. Provisional application Serial No. 60/328,179 (Case 8732P2), filed on Oct. 10, 2001 and U.S. Provisional application Serial No. 60/326,681 (Case 8732P), filed on Oct. 3, 2001 in the name of Midha.

### FIELD

The present invention relates to a hair styling composition that contains particles. More specifically, it relates to a hair styling product that improves hair volume by depositing particles on the hair.

### BACKGROUND

Solid particles are known for use as benefit agents in a variety of formulations and personal care compositions. Solid particles can impart benefits both to the compositions comprising them or surfaces to which the compositions are applied. Solid particles can for example be used as pigments or coloring agents, opacifiers, pearlescent agents, feel modifiers, oil absorbers, skin protectants, matting agents, friction enhancers, slip agents, conditioning agents, exfoliants, odor absorbers, or cleaning enhancers. Additionally, many active ingredients useful as treatment agents for various disorders or socially embarrassing conditions are available and typically used in solid particulate form including antiperspirant agents, anti-dandruff agents, antimicrobials, antibiotics, and sunscreens.

Typically when it is desired to modify the properties of a surface through application of particles, the particles are applied via preparations that are rubbed, sprayed, or otherwise applied directly onto the surface to be affected. Typical personal care preparations suitable for delivery of solid particles to skin surfaces include as examples moisturizers, lotions, and creams. These products are typically applied directly to the surface whereupon particles are deposited and retained by the composition itself or by residual non-volatile elements of the composition after evaporation and drying.

It has also been known to formulate solid particle benefit agents into rinse-off or cleansing compositions such as hair rinses, shampoos, liquid and bar soaps, conditioners, or colorants. Frequently the solid particle benefit agent is used to affect the overall appearance, stability or aesthetics of the composition itself. As example, it is well known to add colorant particles, pigments, or pearlescent agents to compositions to improve the acceptability and attractiveness of the product to potential consumers. It is also well known to add particulate benefit agents to affect the in use performance, appearance or aesthetic properties of the composition or to provide a tactile signal to the user. As example, exfoliant particles are frequently used in cleansing compositions to improve abrasion and removal of oils and dirt from washed surfaces and to impart a perceptible “scrubbing” sensation to the user. Typically such solid particle agents are not intended or desired to be deposited onto the substrate and are removed during dilution and rinsing of the composition from the surface to which they are applied.

Given the broad range of benefits that can be delivered through application and retention of solid particles on surfaces, however, it can be highly desirable to have compositions capable of depositing an effective level of solid particles to the surface treated with compositions containing the desired solid particle benefit agent. Compositions intended to deposit solid particle benefit agents to hair or skin surfaces are known; however, the efficiency of deposition has heretofore been unacceptable, requiring either an excess of the solid particle agent in the composition to affect delivery or an imperceivable or unacceptable level of the benefit to be obtained.

It remains, therefore, highly desirable to have a composition capable of containing and effectively depositing and retaining solid particles on the surface treated therewith.

### SUMMARY

The present invention is directed to a hair styling composition comprising:

(a) from about 0.1% to about 15%, by weight, of an adhesive polymer having a weight average molecular weight of greater than about 20,000;

(b) from about 0.01% to about 20%, by weight, of particles; and

(c) an aqueous carrier.

The present invention is further directed to methods of using the composition.

These and other features, aspects, and advantages of the present invention will become evident to those skilled in the art from a reading of the present disclosure.

### DETAILED DESCRIPTION

While the specification concludes with claims that particularly point out and distinctly claim the invention, it is believed the present invention will be better understood from the following description.

The hair styling compositions of the present invention include an adhesive polymer, particles, and an aqueous carrier. Each of these components, as well as preferred or optional components, is described in detail hereinafter.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified.

All molecular weights as used herein are weight average molecular weights expressed as grams/mole, unless otherwise specified.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “fluid” as used herein, means a liquid or a gas which tends to take the shape of its container, container being the wall of the flexible hollow particles.

The term “flexible” as used herein, means that the hollow particles of the present invention are easy to compress but when pressure is reduced the hollow particles regain their original volume.

The term “fluid-encapsulated” as used herein, means that the hollow particles of the invention are structurally hollow. In accordance with the invention, the term “structurally hollow” nonetheless allows the hollow particles to contain at least one additional material therein.

The term “hollow” as used herein, means a particle having an encapsulated area that is substantially free of solid mass, the encapsulated area comprising from 10 to 99.8 percent of the total volume of the particle.

The term “hydrophobic monomers” as used herein, means monomers that form substantially water insoluble homopolymers.

The term “hydrophilic monomers” as used herein, means monomers that form homopolymers which are substantially water soluble.

The term “permeable” as used herein, means that a substance that permits a liquid or gas to pass through it under given conditions.

The term “polymer” as used herein shall include materials whether made by polymerization of one type of monomer or made by two (i.e., copolymers) or more types of monomers.

The term “solid” as used herein means a particle that is substantially free of voids.

The term “sphere” as used herein, means a spherical body which is the set of points in a metric space whose distance from a fixed point is approximately constant. Here, the meaning of “approximately” is that the fixed points are within a distance of ±15%.

The term “suitable for application to human hair” as used herein, means that the compositions or components thereof so described are suitable for use in contact with human hair and the scalp and skin without undue toxicity, incompatibility, instability, allergic response, and the like.

The term “water soluble” as used herein, means that the polymer is soluble in water in the present composition. In general, the polymer should be soluble at 25° C. at a concentration of 0.1% by weight of the water solvent, preferably at 1%, more preferably at 5%, most preferably at 15%.

All cited references are incorporated herein by reference in their entireties. Citation of any reference is not an admission regarding any determination as to its availability as prior art to the claimed invention.

Particles

The compositions of the present invention include particles. Water insoluble solid particles of various shapes and densities are useful. In a preferred embodiment, the particles tend to have a spherical, an oval, an irregular, or any other shape in which the ratio of the largest dimension to the smallest dimension (defined as the aspect ratio) is less than 10. More preferably, the aspect ratio of the particles is less than 8. Still more preferably, the aspect ratio of the particles is less than 5.

However, it has been found that particles with an aspect ratio of greater than 10 are also useful as long as they remain as aggregated particle stacks or as individual particle stacks on inclusion in an aqueous hair styling composition. Non limiting examples of such particles are Laponite SCPX-2549 and Gelwhite H NF from Southern Clay Products Inc., Flamenco Ultra Silk 2500 and Timica Silkwhite 110W from Engelehard Corp.

Particles useful in the present invention can be natural, synthetic, or semi-synthetic in composition. Hybrid particles are also useful. Synthetic particles can made of either cross-linked or non cross-linked polymers. The particles of the present invention can have surface charges or their surface can be modified with organic or inorganic materials such as surfactants, polymers, and inorganic materials. Particle complexes are also useful.

Non-limiting examples of natural particles include various precipitated silica particles in hydrophilic and hydrophobic forms available from Degussa-Huls under the trade name Sipernet. Snowtex colloidal silica particles available from Nissan Chemical America Corporation.

Examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Other examples include Luna (smooth silica particles) particles available from Phenomenex, MP-2200 (polymethylmethacrylate), EA-209 (ethylene/acrylate copolymer), SP-501(nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful. Synthesis of silicone particles sold under the name Tospearl is generally described in U.S. Pat. Nos. 4,652,618, 4,871,616, and 4,996,257. Depending upon the synthesis conditions the Tospearl particles may have a narrow or a broad size distribution. Particles may also exhibit a spherical surface morphology or an irregular surface morphology. Depending on the surface nature the particles may provide varying level of surface friction on hair fibers. Both the regular and irregular silicone particles are contemplated.

Non-limiting examples of hybrid particles include Ganzpearl GSC-30SR (Sericite & crosslinked polystyrene hybrid powder), and SM-1000, SM-200 (mica and silica hybrid powder available from Presperse).

In one embodiment of the present invention, the particles used in the hair styling composition are hollow particles. In a preferred embodiment, the hollow particles are fluid-encapsulated, flexible microspheres. The microspheres are structurally hollow, however, they may contain various fluids, which encompass liquids and gases and their isomers. The gases include, but not limited to, butane, pentane, air, nitrogen, oxygen, carbon dioxide, and dimethyl ether. If used, liquids may only partially fill the microspheres. The liquids include water and any compatible solvent. The liquids may also contain vitamins, amino acids, proteins and protein derivatives, herbal extracts, pigments, dyes, antimicrobial agents, chelating agents, UV absorbers, optical brighteners, silicone compounds, perfumes, humectants which are generally water soluble, additional conditioning agents which are generally water insoluble, and mixtures thereof. In one embodiment, water-soluble components are preferred encompassed material. In another embodiment, components selected from the group consisting of vitamins, amino acids, proteins, protein derivatives, herbal extracts, and mixtures thereof are preferred encompassed material. In yet another embodiment, components selected from the group consisting of vitamin E, pantothenyl ethyl ether, panthenol, Polygonum multiflori extracts, and mixtures thereof are preferred encompassed material.

The particles of the present invention can have surface charges or their surface can be modified with organic or inorganic materials such as surfactants, polymers, and inorganic materials. Particle complexes are also useful. Non-limiting examples of complexes of gas-encapsulated microspheres are DSPCS-12™ (silica modified ethylene/methacrylate copolymer microsphere) and SPCAT-2™ (talc modified ethylene/methacrylate copolymer microsphere). Both of these are available from Kobo Products, Inc.

The surface of the particle may be charged through a static development or with the attachment of various ionic groups directly or linked via short, long or branched alkyl groups. The surface charge can be anionic, cationic, zwitterionic or amphoteric in nature.

The wall of the particles of the present invention may be formed from a thermoplastic material. The thermoplastic material may be a polymer or copolymer of at least one monomer selected from the following groups: acrylates, methacrylates, styrene, substituted styrene, unsaturated dihalides, acrylonitriles, methacrylonitrile. The thermoplastic materials may contain amide, ester, urethane, urea, ether, carbonate, acetal, sulfide, phosphate, phosphonate ester, and siloxane linkages. The hollow particles may comprise from 1% to 60% of recurring structural units derived from vinylidene chloride, from 20% to 90% of recurring structural units derived from acrylonitrile and from 1% to 50% of recurring structural units derived from a (meth)acrylic monomer, the sum of the percentages (by weight) being equal to 100. The (meth)acrylic monomer is, for example, a methyl acrylate or methacrylate, and especially the methacrylate. Preferably, the particles are comprised of a polymer or copolymer of at least one monomer selected from expanded or non-expanded vinylidene chloride, acrylic, styrene, and (meth)acrylonitrile. More preferably, the particles are comprised of a copolymer of acrylonitrile and methacrylonitrile.

Particles comprised of polymers and copolymers obtained from esters, such as, for example, vinyl acetate or lactate, or acids, such as, for example, itaconic, citraconic, maleic or fumaric acids may also be used. See, in this regard, Japanese Patent Application No. JP-A-2-112304, the full disclosure of which is incorporated herein by reference.

Non-limiting examples of commercially available suitable particles are 551 DE (particle size range of approximately 30-50 μm and density of approximately 42 kg/m³), 551 DE 20 (particle size range of approximately 15-25 μm and density of approximately 60 kg/m³), 461 DE (particle size range of approximately 20-40 μm and density 60 kg/m³), 551 DE 80 (particle size of approximately 50-80 μm and density of approximately 42 kg/m³), 091 DE (particle size range of approximately 35-55 μm and density of approximately 30 kg/m³), all of which are marketed under the trademark EXPANCEL™ by Akzo Nobel. Other examples of suitable particles for use herein are marketed under the trademarks DUALITE® and MICROPEARL™ series of microspheres from Pierce & Stevens Corporation. Particularly preferred hollow particles are 091 DE and 551DE 50. The hollow particles of the present invention exist in either dry or hydrated state. The aforesaid particles are nontoxic and non-irritating to the skin.

Hollow particles that are useful in the invention can be prepared, for example, via the processes described in EP-56,219, EP-348,372, EP-486,080, EP-320,473, EP-112,807 and U.S. Pat. No. 3,615,972, the full disclosure of each of which is incorporated herein by reference.

Alternatively, the wall of the hollow particles useful in the present invention may be formed from an inorganic material. The inorganic material may be a silica, a soda-lime-borosilicate glass, a silica-alumina ceramic, or an alkali alumino silicate ceramic. Non-limiting examples of commercially available suitable low density, inorganic particles are H50/10,000 EPX (particle size range approximately 20-60 μm), S38 (particle size range approximately 15-65 μm), W-210 (particle size range approximately 1-12 μm), W410 (particle size range approximately 1-24 μm), W-610 (particle size range approximately 1-40 μm), G-200 (particle size range approximately 1-12 μm), G400 (particle size range approximately 1-24 μm), G-600 (particle size range approximately 1-40 μm), all of which are marketed under the trademarks 3M™ Scotchlite™ Glass Bubbles, 3M™ Zeeospheres™ ceramic microspheres, and 3M™ Z-Light Spheres™ Ceramic Microspheres. Also useful are Silica shells (average particle size 3 μm) available from KOBO Products and LUXSIL™ (3-13 μm mean diameter) available from PQ Corporation.

The particles of the present invention preferably have a particle size of 0.1 μm or greater. Preferably, the particles have a particle size of greater than about 0.5 μm. Preferably, the particles have a particle size of less than about 80 μm in diameter. More preferably, the particles range from about 1 μm to about 70 μm, still more preferably from about 2 μm to about 65 μm, and even more preferably from about 2 μm to about 60 μm in diameter.

Preferably, the wall of the hollow particles useful in the invention is flexible. “Flexible”, as used herein, means that the hollow particles are easy to compress. When pressure is reduced the hollow particles regain their original volume. The flexible hollow particles could alter their shape under an applied stress, or thermal expansion and contraction due to temperature change. Thus, the particles could expand upon heating.

The particles of the invention may be permeable or non-permeable. “Permeable”, as used herein, means that they permit a liquid or gas to pass through them under given conditions. Preferably, a majority of the particles of the present invention will maintain their structural integrity during normal use of the hair styling composition. More preferably, substantially all of the particles maintain their structural integrity during normal use of the hair styling composition.

Preferred particles will also have physical properties which are not significantly affected by typical processing of the composition. Preferably, particles having melting points greater than about 70° C. are used. Still more preferably, particles having a melting point greater than 80° C. are used and most preferrably particles having melting point of greater than about 95° C. are used. As used herein, melting point would refer to the temperature at which the particle transitions to a liquid or fluid state or undergoes significant deformation or physical property changes. In addition, many of the particles of present invention are cross-linked or have a cross-linked surface membrane. These particles do not exhibit a distinct melting point. Cross-linked particles are also useful as long as they are stable under the processing and storage conditions used in the making of compositions.

The compositions of the present invention comprise at least 0.01% by weight of particles. Preferably, the compositions of the present invention include at least 0.025% by weight of particles, more preferably at least 0.1%, still more preferably at least 0.2%, and even more preferably at least 0.5% by weight of hollow particles. In the compositions of the present invention, it is preferable to incorporate no more than about 20% by weight of particles, more preferably no more than about 10%, still more preferably no more than 5%, and even more preferably no more than 2% by weight of particles.

Aqueous Carrier

The compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources containing mineral cations can also be used, depending on the desired characteristic of the product. Generally, the compositions of the present invention comprise from about 20% to about 99%, preferably from about 40% to about 98%, and more preferably from about 60% to about 98% aqueous carrier.

The pH of the present composition is preferably from about 4 to about 9, more preferably from about 4.5 to about 7.5. Buffers and other pH adjusting agents can be included to achieve the desirable pH.

Adhesive Polymer

The compositions of the present invention comprise an adhesive polymer. The compositions hereof will generally comprise from about 0.1% to about 15%, preferably from 0.5% to about 8%, more preferably from about 1% to about 8%, by weight of the composition, of the adhesive polymer. It is not intended to exclude the use of higher or lower levels of the polymers, as long as an effective amount is used to provide adhesive or film-forming properties to the composition and the composition can be formulated and effectively applied for its intended purpose. By adhesive polymer what is meant is that when applied as a solution to a surface and dried, the polymer forms a film. Such a film will have adhesive and cohesive strength, as is understood by those skilled in the art.

These styling polymers provide the composition of the present invention with hair styling performance by providing polymeric deposits on the hair after application. The polymer deposited on the hair has adhesive and cohesive strength and delivers styling primarily by forming welds between hair fibers upon drying, as is understood by those skilled in the art.

Many such polymers are known in the art, including water-insoluble organic polymers and water-insoluble silicone-grafted polymers, all of which are suitable for use in the composition herein, provided that they also have the requisite features or characteristics described hereinafter. Such polymers can be made by conventional or otherwise known polymerization techniques well known in the art, an example of which includes free radical polymerization.

The adhesive polymer will preferably be a carbon chain derived from polymerization of ethylenically unsaturated monomers, but can also be, cellulosic chains or other carbohydrate-derived polymeric chains. The backbone can also include ether groups, ester groups, amide groups, urethanes and the like.

The adhesive polymer should have a weight average molecular weight of at least about 20,000, preferably greater than about 25,000, more preferably greater than about 30,000, most preferably greater than about 35,000. There is no upper limit for molecular weight except that which limits applicability of the invention for practical reasons, such as processing, aesthetic characteristics, ability to formulate, etc. In general, the weight average molecular weight will be less than about 10,000,000, more generally less than about 5,000,000, and typically less than about 2,000,000. Preferably, the weight average molecular weight will be between about 20,000 and about 2,000,000, more preferably between about 30,000 and about 1,000,000, and most preferably between about 40,000 and about 500,000.

Preferably, the adhesive hereof when dried to form a film have a Tg or Tm of at least about −20° C., more preferably at least about 20° C., so that they are not unduly sticky, or “tacky” to the touch. As used herein, the abbreviation “Tg” refers to the glass transition temperature of the backbone of the polymer, and the abbreviation “Tm” refers to the crystalline melting point of the backbone, if such a transition exists for a given polymer. Preferably, both the Tg and the Tm, if any, are above about −20° C., more preferably above about 20° C.

The adhesive polymer monomer units can be derived from polar, or hydrophilic, monomers, “A” monomers, low polarity, or hydrophobic, “B” monomers, or mixtures of polar hydrophilic “A” monomers and low polarity, hydrophobic, “B” monomers.

“Hydrophobic monomers” means monomers which form substantially water insoluble homopolymers. “Hydrophilic monomers” means monomers which form homopolymers which are substantially water soluble. Substantially water soluble shall refer to monomers that form homopolymers that are soluble in distilled (or equivalent) water, at 25° C., at a concentration of 0.2% by weight, and are preferably soluble at 1.0% by weight. Substantially water insoluble shall refer to monomers that form homopolymers that are not soluble in distilled (or equivalent) water, at 25° C., at a concentration of 0.2% by weight, and preferably not soluble at 0.1% by weight. The weight average molecular weight for purposes of determining substantial water solubility or insolubility shall be about 40,000, although solubility at higher molecular weight shall also be indicative of solubility at about 40,000.

The particular relative amounts of A and B monomers can vary as long as the polymer is soluble in the solvent system hereof.

Representative examples of A monomers include acrylic acid, methacrylic acid, N,N-dimethylacrylamide, dimethyl aminoethyl methacrylate, quaternized dimethylaminoethyl methacrylate, methacrylamide, N-t-butyl acrylamide, maleic acid, maleic anhydride and its half esters, crotonic acid, itaconic acid, acrylamide, acrylate alcohols, hydroxyethyl methacrylate, diallyldimethyl ammonium chloride, vinyl pyrrolidone, vinyl ethers (such as methyl vinyl ether), maleimides, vinyl pyridine, vinyl imidazole, other polar vinyl heterocyclics, styrene sulfonate, allyl alcohol, vinyl alcohol (such as that produced by the hydrolysis of vinyl acetate after polymerization), salts of any acids and amines listed above, and mixtures thereof. Preferred A monomers include acrylic acid, N,N-dimethyl acrylamide, dimethylaminoethyl methacrylate, quaternized dimethyl aminoethyl methacrylate, vinyl pyrrolidone, salts of acids and amines listed above, and mixtures thereof.

Representative examples of B monomers are acrylic or methacrylic acid esters of C₁-C₁₈ alcohols, such as methanol, ethanol, methoxy ethanol, 1-propanol, 2-propanol, 1-butanol, 2-methyl-1-propanol, 1-pentanol, 2-pentanol, 3-pentanol, 2-methyl-1-butanol, 1-methyl-1-butanol, 3-methyl-1-butanol, 1-methyl-1-pentanol, 2-methyl-1-pentanol, 3-methyl-1-pentanol, t-butanol(2-methyl-2-propanol), cyclohexanol, neodecanol, 2-ethyl-1-butanol, 3-heptanol, benzyl alcohol, 2-octanol, 6-methyl-1-heptanol, 2-ethyl-1-hexanol, 3,5-dimethyl-1-hexanol, 3,5,5-tri methyl-1-hexanol, 1-decanol, 1-dodecanol, 1-hexadecanol, 1-octa decanol, and the like, the alcohols having from about 1-18 carbon atoms with the number of carbon atoms preferably being from about 1-12; styrene; polystyrene macromer; vinyl acetate; vinyl chloride; vinylidene chloride; vinyl propionate; alpha-methylstyrene; t-butylstyrene; butadiene; cyclohexadiene; ethylene; propylene; vinyl toluene; and mixtures thereof. Preferred B monomers include n-butyl methacrylate, isobutyl methacrylate, t-butyl acrylate, t-butyl methacrylate, 2-ethylhexyl methacrylate, methyl methacrylate, and mixtures thereof. Most preferably, B is selected from t-butyl acrylate, t-butyl methacrylate, and mixtures thereof. The level of A monomer units can be from about 0% to about 100%, preferably from about 5% to about 80%, more preferably from about 10% to about 50%, most preferably from about 15% to about 40%; the level of B monomer units, can be from 0% to about 100%, preferably from about 20% to about 95%, more preferably from about 50% to about 90%, most preferably from about 60% to about 85%.

The composition of any particular adhesive polymer will help determine its formulational properties. By appropriate selection and combination of particular A and B components, the adhesive polymer can be optimized for inclusion in specific solvent vehicles. The adhesive polymer included in the compositions hereof must be soluble in the polar solvent. This is determined according to whether the polymer can stay in solution or precipitates out of solution at 25° C. at the concentration present in the composition. It is well within the skill of one in the art to select monomers for incorporation into the polymers for formulateability and solubility in selected solvent systems.

Exemplary adhesive polymers for use in the present invention include the following, where the numbers following the structure indicate the weight ratios of monomers as loaded into the polymerization reactor:

(i) acrylic acid/t-butyl acrylate 25/75

(ii) dimethylaminoethyl methacrylate/isobutyl methacrylate/2-ethylhexyl-methacrylate 40/40/20

(iii) t-butylacrylate/acrylic acid 65/35

(iv) polymer (ii) quaternized by treatment with methyl choride

The adhesive polymers can be synthesized by free radical polymerization of the monomers. The general principles of free radical polymerization methods are well understood. See, for example, Odian, “Principles of Polymerization”, 3nd edition, John Wiley & Sons, 1991, pp. 198-334. The desired monomers are all placed in a reactor, along with a sufficient amount of a mutual solvent so that when the reaction is complete the viscosity of the reaction is reasonable. Typical monomer loadings are from about 20% to about 50%. Undesired terminators, especially oxygen, are removed as needed. This is done by evacuation or by purging with an inert gas, such as argon or nitrogen. The initiator is introduced and the reaction brought to the temperature needed for initiation to occur, assuming thermal initiators are used. Alternatively, redox or radiation initiation can be used. The polymerization is allowed to proceed as long as needed for a high level of conversion to be achieved, typically from a few hours to a few days. The solvent is then removed, usually by evaporation or by precipitating the polymer by addition of a nonsolvent. The polymer can be further purified, as desired.

As an alternative to a batch reaction, the adhesive polymer can be made by a semi-continuous or continuous process. In the semi-continuous process, two or more additions of monomers are made during the polymerization reaction. This is advantageous when the polymer is made of several monomers which react during the polymerization at different rates. The proportions of monomers added to the reaction at the separate points of addition can be adjusted by one of ordinary skill in the art such that the polymers of the final product have a more uniform structure. In other words, the polymers of the final product will have a more consistent monomer content distribution for each of the monomer types charged to the reaction.

As is known in the art, polymers which have acidic functionalities, such as carboxyl groups, are usually used in at least partially neutralized form to promote solubility/dispersibility of the polymer. In addition, use of the neutralized form aids in the ability of the hair care compositions to be removed from the hair by shampooing. In general, it is preferred that from about 10% to 100%, more preferably from about 20% to about 90%, and most more preferably from about 40% to about 85%, of the acidic monomers of the polymer be neutralized.

Any conventionally used base, organic or metallic, may be used for neutralization of the polymers. Metallic bases are particularly useful in the present compositions. Hydroxides, where the cation is an alkali metal or an alkaline earth metal, are suitable neutralizers for use in the present hair spray compositions.

Preferred neutralizing agents for use in hair spray compositions of the present invention are potassium hydroxide and sodium hydroxide.

Examples of other suitable neutralizing agents which may be included in the hair spray compositions of the present invention include amines, especially amino alcohols such as 2-amino-2-methyl-1,3-propanediol (AMPD), 2-amine-2-ethyl-1,3-propanediol (AEPD), 2-amino-2-methyl-1-propanol (AMP), 2-amino-1-butanol (AB), monoethanolamine (MEA), diethanolamine (DEA), triethanolamine (TEA), monoisopropanolamine (MIPA), di-isopropanol-amine (DIPA), tri-isopropanolamine (TIPA) and dimethyl steramine (DMS). Particularly useful neutralizing agents are mixtures of amines and metallic bases.

Polymers having basic functionalities, e.g., amino groups, are preferably at least partially neutralized with an acid, e.g., hydrogen chloride.

Solubility of the adhesive polymer, as described above, should be determined after neutralization, if any, as well as after addition of other ingredients that may be included in the polar solvent phase, such as surfactants, solubilizers, etc.

### Silicone Grafted Adhesive Polymers

Other useful adhesive polymers include silicone-containing hair styling resins. Preferably, the silicone-containing hair styling resin is compatible with the adhesive polymer described above. This silicone containing hair styling resin is preferably colloidally dispersed or solubilized in the hair cosmetic carrier along with the adhesive hair styling polymer. Keeping the two hair styling agents dispersed and solubilised in the hair spray solvent is believed to be important for providing the unique hair setting benefits in combination with excellent hair feel characteristics which are delivered by compositions according to the present invention.

The compositions hereof may generally comprise from about 0.1% to about 15%, preferably from 0.5% to about 8%, more preferably from about 1% to about 8%, by weight of the composition, of the silicone grafted polymer. It is not intended to exclude the use of higher or lower levels of the polymers, as long as an effective amount is used to provide adhesive or film-forming properties to the composition and the composition can be formulated and effectively applied for its intended purpose. By adhesive polymer what is meant is that when applied as a solution to a surface and dried, the polymer forms a film. Such a film will have adhesive and cohesive strength, as is understood by those skilled in the art.

The silicone grafted polymers are characterized by polysiloxane moieties covalently bonded to and pendant from a polymeric carbon-based backbone. The polymeric backbone is chosen such that it is compatible with the non-silicone adhesive styling polymer. By “compatible” is meant is that, when placed in a suitable solvent, the polymers form a stable solution, i.e., the polymers do not compete for solubility and therefore, cause no phase separation and when the solution is dried a uniform film is formed, with no macrophase separation of the two polymers. A suitable solvent is a solvent which substantially completely dissolves the non-silicone and silicone grafted polymers at the levels described herein. The polymer blend forms a relatively clear hairspray system (% transmittance at 450 nm is generally greater than 80%). It is recognized that certain plasticizers can form cloudy films as well as incorrect neutralization levels. Therefore, this would fall outside this definition of compatibility. The compatibility can be tested by dissolving the adhesive polymer and the silicone grafted hair styling resin in a mutual solvent, and then evaporating the solvent to form a film. Incompatible polymers will form a cloudy film with poor mechanical properties, due to the large scale phase separation of the two polymers. Alternatively, after drying the polymer solution to a film, compatibility can be evaluated by measuring the Tg. Compatible polymers will have a single Tg, while incompatible polymers will exhibit two Tg's. Although compatibility can occur between two polymers of completely different structures, it is preferred that compatibility be obtained by making the composition of the non-silicone backbone of the silicone grafted polymer similar to or identical to the composition of the adhesive polymer.

The backbone will preferably be a carbon chain derived from polymerization of ethylenically unsaturated monomers, but can also be cellulosic chains or other carbohydrate-derived polymeric chains to which polysiloxane moieties are pendant. The backbone can also include ether groups, ester groups, amide groups, urethane groups and the like. The polysiloxane moieties can be substituted on the polymer or can be made by co-polymerization of polysiloxane-containing polymerizable monomers (e.g. ethylenically unsaturated monomers, ethers, and/or epoxides) with non-polysiloxane-containing polymerizable monomers.

The polysiloxane-grafted polymer should have a weight average molecular weight of at least about 20,000. There is no upper limit for molecular weight except that which limits applicability of the invention for practical reasons, such as processing, aesthetic characteristics, formulateability, etc. In general, the weight average molecular weight will be less than about 10,000,000, more generally less than about 5,000,000, and typically less than about 3,000,000. Preferably, the weight average molecular weight will be between about 50,000 and about 2,000,000, more preferably between about 75,000 and about 1,000,000, most preferably between about 100,000 and about 750,000.

Preferably, the adhesive hereof when dried to form a film have a Tg or Tm of at least about −20° C., more preferably at least about 20° C., so that they are not unduly sticky, or “tacky” to the touch. As used herein, the abbreviation “Tg” refers to the glass transition temperature of the backbone of the polymer, and the abbreviation “Tm” refers to the crystalline melting point of the backbone, if such a transition exists for a given polymer. Preferably, both the Tg and the Tm, if any, are above about −20° C., more preferably above about 20° C.

The silicone grafted polymers for the compositions of the present invention comprise “silicone-containing” (or “polysiloxane-containing”) monomers, which form the silicone macromer pendant from the backbone, and non-silicone-containing monomers, which form the organic backbone of the polymer.

The silicone grafted polymers should satisfy the following three criteria:

(1) when dried, either alone or in the presence of the adhesive polymer, the polymer phase-separates into a discontinuous phase which includes the polysiloxane portion and a continuous phase which includes the non-polysiloxane portion;

(2) the polysiloxane portion is covalently bonded to the non-polysiloxane portion; and

(3) the molecular weight of the polysiloxane portion is at least about 500.

When used in a composition, such as a personal care composition for application to the hair or skin, the non-polysiloxane portion should permit the polymer to deposit on the intended surface, such as hair or skin.

Without being limited by theory, it is believed that the phase separation property provides a specific orientation of the polymer which results in the desired combination of tactile feel, and film-forming or adhesive benefits. The phase-separating nature of the compositions of the present invention may be determined as follows:

The polymer is cast as a solid film out of a solvent (i.e., a solvent which dissolves both the backbone and the polysiloxane-graft portions). This film is then sectioned and examined by transmission electron microscopy. Microphase separation is demonstrated by the observation of inclusions in the continuous phase. These inclusions should have the proper size to match the size of the silicone chain (typically a few hundred nm or less) and the proper density to match the amount of silicone present. This behavior is well documented in the literature for polymers with this structure (see, for example, S. D. Smith, Ph.D. Thesis, University of Virginia, 1987, and references cited therein, said thesis incorporated by reference herein).

A second method for determining phase-separating characteristics involves examining the enrichment of the concentration of silicone at the surface of a polymer film relative to the concentration in the bulk polymer. Since the silicone prefers the low energy air interface, it preferentially orients on the polymer surface. This produces a surface with the silicone oriented at the surface of the film. This can be demonstrated experimentally by ESCA (electron spectroscopy for chemical analysis) of the dried film surface. Such an analysis shows a high level of silicone and a greatly reduced level of backbone polymer when the film surface is analyzed. (Surface here means the first few tens of Angstroms of film thickness.) By varying the angle of the interrogating beam the surface can be analyzed to varying depths.

The preferred silicone grafted polymers comprise an organic backbone preferably a carbon backbone derived from ethylenically unsaturated monomers, such as a vinyl polymeric backbone, and a polysiloxane macromer (especially preferred are polydialkylsiloxane, most preferably polydimethylsiloxane) grafted to the backbone. The polysiloxane macromer should have a weight average molecular weight of at least about 500, preferably from about 1,000 to about 100,000, more preferably from about 2,000 to about 50,000, most preferably about 5,000 to about 20,000. Organic backbones contemplated include those that are derived from polymerizable, ethylenically unsaturated monomers, including vinyl monomers, and other condensation monomers (e.g., those that polymerize to form polyamides and polyesters), ring-opening monomers (e.g., ethyl oxazoline and caprolactone), etc. Also contemplated are backbones based on cellulosic chains, ether-containing backbones, etc.

Preferably the weight ratio of the non-silicone polymer to silicone grafted polymer ranges from about 1:10 to about 1:1, preferably from about 1:5 to about 1:1.

Examples of useful polymers and how they are made are described in detail in U.S. Pat. No. 4,693,935, Mazurek, issued Sep. 15, 1987, U.S. Pat. No. 4,728,571, Clemens et al., issued Mar. 1, 1988, both of which are incorporated herein by reference.

Suitable silicone grafted polymers are also disclosed in EPO Application 90307528.1, published as EPO Application 0 408 311 A2 on Jan. 11, 1991, Hayama, et al., U.S. Pat. No. 5,061,481, issued Oct. 29, 1991, Suzuki et al., U.S. Pat. No. 5,106,609, Bolich et al., issued Apr. 21, 1992, U.S. Pat. No. 5,100,658, Bolich et al., issued Mar. 31, 1992, U.S. Pat. No. 5,100,657, Ansher-Jackson, et al., issued Mar. 31, 1992, U.S. Pat. No. 5,104,646, Bolich et al., issued Apr. 14, 1992, U.S. Ser. No. 07/758,319, Bolich et al, filed Aug. 27, 1991, and U.S. Ser. No. 07/758,320, Torgerson et al., filed Aug. 27, 1991, all of which are incorporated by reference herein.

The preferred silicone grafted polymers are comprised of monomer units derived from: at least one free radically polymerizable ethylenically unsaturated monomer or monomers and at least one free radically polymerizable polysiloxane-containing ethylenically unsaturated monomer or monomers.

The silicone grafted polymers hereof generally comprise from about 1% to about 50%, by weight, of polysiloxane-containing monomer units, i.e., monomer units polysiloxane-containing monomers (referred to herein as “C” monomers), and from about 50% to about 99% by weight, of non-polysiloxane-containing monomers.

The non-polysiloxane-containing monomer units can be derived from polar, or hydrophilic, monomers, “A” monomers, or mixtures of polar hydrophilic monomers, low polarity, or hydrophobic, “B” monomers or mixtures of the two. The definitions of the “A” and “B” monomers, and representative examples of the “A” and “B” monomers, are the same as those use for the “A” and “B” monomers in the adhesive polymer.

The particular relative amounts of A, B, and C monomers can vary as long as the polymer backbone is soluble in the polar solvent hereof, the polymer backbone is compatible with the adhesive polymer, and the silicone grafted copolymer exhibits phase separation when dried.

Polymerizable polysiloxane-containing monomers (C monomer) are exemplified by the general formula:

X(Y)ₙSi(R)₃₋ₘZₘ

wherein X is an ethylenically unsaturated group copolymerizable with the A and B monomers, such as a vinyl group; Y is a divalent linking group; R is a hydrogen, hydroxyl, lower alkyl (e.g. C₁-C₄), aryl, alkaryl, alkoxy, or alkylamino; Z is a monovalent siloxane polymeric moiety having a number average molecular weight of at least about 500, is essentially unreactive under copolymerization conditions, and is pendant from the vinyl polymeric backbone described above; n is 0 or 1; and m is an integer from 1 to 3. C has a weight average molecular weight as described above. Preferably, the C monomer has a formula selected from the following group:

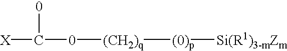

In this structure, m is 1, 2 or 3 (preferably m=1); p is 0 or 1; q is an integer from 2 to 6; R¹ is hydrogen, hydroxyl, lower alkyl, alkoxy, alkylamino, aryl, or alkaryl (preferably R¹ is alkyl); X is

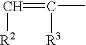

R² is hydrogen or —COOH (preferably R² is hydrogen); R³ is hydrogen, methyl or —CH₂COOH (preferably R³ is methyl); Z is

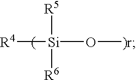

R⁴, R⁵, and R⁶ independently are lower alkyl, alkoxy, alkylamino, aryl, arkaryl, hydrogen or hydroxyl (preferably R⁴, R⁵, and R⁶ are alkyls); and r is an integer of about 5 or higher, preferably about 10 to about 1500 (most preferably r is from about 100 to about 250). Most preferably, R⁴, R⁵, and R⁶ are methyl, p=0, and q=3. Also preferred, the C monomer has the formula selected from the group:

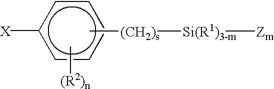

or

X-CH₂—(CH₂)_{S}—Si(R¹)₃₋ₘ-Zₘ

wherein: s is an integer from 0 to about 6, preferably 0, 1, or 2, more preferably 0 or 1; m is an integer from 1 to 3, preferably 1; R² is C1-C10 alkyl or C7-C10 alkylaryl, preferably C1-C6 alkyl or C7-C10 alkylaryl, more preferably C1-C2 alkyl; n is an integer from 0 to 4, preferably 0 or 1, more preferably 0.

In general, the silicone, grafted polymer will preferably comprise from about 50% to about 99%, more preferably from about 60% to about 98%, most preferably from about 75% to about 95%, by weight of the polymer, of non-silicone macromer-containing monomer units, e.g. the total A and B monomer units, and from about 1% to about 50%, preferably from about 2% to about 40%, more preferably from about 5% to about 25%, of silicone macromer-containing monomer units, e.g. the C monomer units. The level of A monomer units can be from about 0% to about 99%, preferably from about 5% to about 80%, more preferably from about 10% to about 50%, most preferably from about 15% to about 40%; the level of B monomer units, can be from 0% to about 99%, preferably from about 1% to about 90%, more preferably from about 5% to about 85%, most preferably from about 15% to about 80%; and the level of C monomer units, from about 1% to about 50%, preferably from about 2% to about 40%, more preferably from about 5% to about 25%.

The combination of the A and B monomers preferably comprises from about 50.0% to about 99.9% (more preferably about 60% to about 99%, most preferably from about 75% to about 95%) of the polymer. The composition of any particular copolymer will help determine its formulational properties. In fact, by appropriate selection and combination of particular A, B and C components, the copolymer can be optimized for inclusion in specific vehicles. For example, polymers which are soluble in an aqueous formulation preferably have the composition: from about 0% to about 70% (preferably from about 5% to about 70%) monomer A, from about 30% to about 98% (preferably from about 30% to about 80%) monomer B, and from about 1% to about 40% monomer C. Polymers which are dispersible have the preferred composition: from about 0% to about 70% (more preferably from about 5% to about 70%) monomer A, from about 20% to about 80% (more preferably from about 20% to about 60%) monomer B, and from about 1% to about 40% monomer C.

The composition of any particular silicone grafted polymer will help determine its formulational properties. By appropriate selection and combination of particular A, B and C components, the silicone grafted polymer can be optimized for inclusion in specific vehicles. The backbone of the silicone grafted polymer included in the compositions hereof must be soluble in the polar solvent, which is hereinafter referred to as the silicone grafted polymer, as a whole, being soluble in the polar solvent. This is determined according to whether the polymer can stay in solution or precipitates out of solution at 25° C. at the concentration present in the composition or whether the range of concentrations for silicone grafted polymer discribed herein. It is well within the skill of one in the art to select monomers for incorporation into the polymers for formulateability and solubility in selected polar solvent systems.

Exemplary silicone grafted polymers for use in the present invention include the following, where the composition is given as weight part of monomer used in the synthesis:

(i) acrylic acid/n-butylmethacrylate/polydimethylsiloxane (PDMS) macromer 20,000 molecular weight macromer 20/70/10

(ii) dimethylaminoethyl methacrylate/isobutyl methacrylate/2--ethylhexyl-methacrylate/PDMS macromer-20,000 molecular weight macromer 40/30/15/15

(iii) t-butylacrylate/acrylic acid/PDMS macromer-10,000 molecular weight macromer 63.5/20/16.5

(iv) t-butylacrylate/acrylic acid/PDMS macromer-20,000 molecular weight macromer 60/20/20

The silicone grafted polymers can be synthesized by free radical polymerization of the polysiloxane-containing monomers with the non-polysiloxane-containing monomers. The synthetic procedures are in general the same as those described for the adhesive copolymer. The silicone macromer is added in to the reactor along with the “A” and “B” monomers, and the reaction proceeds as for the adhesive copolymer examples. Compared to the adhesive copoymer, it may be necessary to choose different solvents for the polymerization reaction, as apparent to one skilled in the art, to keep the monomers and polymers in solution thrroughout the polymerization.

Without being limited by theory, it is believed that in forming the above-described silicone grafted polymers, there is some polymer which does not encorporate the silicone graft; such polymers have a relatively low weight average molecular weight e.g., below 20,000.

### Cationic Adhesive Copolymers

Suitable cationic polymers include Polyquaternium-4 (Celquat H-100; L200—supplier National Starch); Polyquaternium-10 (Celquat SC-240C; SC-230 M—supplier National Starch); (UCARE polymer series—JR-125, JR-400, LR-400, LR-30M, LK, supplier Amerchol); Polyquaternium-11 (Gafquat 734; 755N—supplier ISP); Polyquaternium-16 (Luviquat FC 370; FC550; FC905; HM-552 supplier by BASF); PVP/Dimethylaminoethylmethacrylate (Copolymer 845; 937; 958-ISP supplier); Vinyl Caprolactam/PVP/Dimethylaminoethyl Methacrylate copolymer (Gaffix VC-713; H20LD EP-1—supplier ISP); Chitosan (Kytamer L; Kytamer PC—supplier Amerchol); Polyquaternium-7 (Merquat 550—supplier Calgon); Polyquaternium-18 (Mirapol AZ-1 supplied by Rhone-Poulenc); Polyquaternium-24 (Quatrisoft Polymer LM-200—supplier Amerchol); Polyquaternium-28 (Gafquat HS-100—supplier ISP); Polyquaternium-46 (Luviquat Hold—supplier BASF);and Chitosan Glycolate (Hydagen CMF; CMFP—supplier Henkel); Hydroxyethyl Cetyldimonium Phosphate (Luviquat Mono CP—supplier BASF); and Guar Hydroxylpropyl Trimonium Chloride (Jaguar C series-13S, -14S, -17, 162,-2000, Hi-CARE 1000—supplier Rhône-Poulenc).

Suitable amphoteric polymers include Octylacrylmide/Acrylates/Butylaminoethyl Methacrylate Copolymer (Amphomer 284910, Amphomer LV-71 284971, Lovocryl-47 28-4947—National Starch supplier), and Methacryloyl ethyl betaine/methacrylates copolymer (Diaformer series supplier Mitsubishi).

Polymers which are partially zwitterionic are also useful. They possess a positive charge over a broad range of pH but contain acidic groups which are only negatively charged at basic pH. The polymer is positively charged at lower pH and neutral (have both negative and positive charge) at higher pHs. The zwitterionic polymer may be selected from cellulose derivatives, wheat derivatives and chitin derivatives such as are known in the art. Nonlimiting examples of zwitterionic polymers useful herein include Polyquaternium47 (Merquat 2001—supplier Calgon (a zwitterionic copolymer of acrylic acid, methacryl amido propyl trimethyl ammonium chloride, and methyl acrylate)); Carboxyl Butyl Chitosan (Chitolam NB/101—marketed by Pilot Chemical Company, developed by Lamberti); and Dicarboxyethyl Chitosan (N-[(3′-hydroxy-2′,3′-dicarboxy)ethyl]-beta-D-(1,4)-glucosamine) (available from Amerchol as, e.g., CHITOLAM NB/101).

Useful nonionic polymers include PVP or Polyvinylpyrrolidone (PVP K-15, K-30, K-60, K-90, K-120—supplier ISP) (Luviskol K series 12, 17, 30, 60, 80, & 90—supplier BASF); PVP/VA (PVP/VA series S-630; 735, 635, 535, 335, 235—supplier ISP)(Luviskol VA); PVP/DMAPA acrylates copolymer (Styleze CC-10—supplier ISP); PVP/VA/Vinyl Propionate copolymer (Luviskol VAP 343 E, VAP 343 I, VAP 343 PM—supplier BASF); Hydroxylethyl Cellulose (Cellosize HEC—supplier Amerchol); and Hydroxylpropyl Guar Gum (Jaguar HP series-8, -60, -105, -120—supplier Rhône-Poulenc).

### Other Adhesive Polymers

Polyol copolymers may be useful as adhesive polymers in the present invention. The polyol copolymers comprise a liquid or semisolid hair styling agent suitable for being left on dry hair as a liquid or semisolid after the composition has been applied and allowed to dry on the hair. These hair styling agents provide for a fluid film to be left on the hair which can be characterized as a reformable weld that provides dry hair restyling performance without the need to reapply the compositions or add additional styling aids on the hair.

The concentration of the polyol copolymer may vary with each selected hair styling formulation, but such concentrations will generally range from about 3% to about 50%, more preferably from about 5% to about 25%, even more preferably from about 7% to about 15%, by weight of the composition.

Suitable styling agents for use in the compositions of the present invention include any known or otherwise effective hair styling agents, other than polyalkylene glyceryl ethers, that are liquids or semisolids under ambient conditions and that can remain a liquid or semisolid after the composition has been applied and allowed to dry on dry hair. It has been found that certain liquid or semisolid styling agents, particularly low molecular weight polyalkylene glycols, can leave a fluid film on the hair that allows the hair fibers to be separated by forces such as wind, and then re-adhere using styling techniques such as combing, brushing, or running your fingers through the hair. This separation/readherence property provided by the styling agents defined herein results in improved dry hair restyling performance for several days without leaving the hair feeling unduly sticky or stiff, and without having to reapply the compositions described herein and/or add any other additional styling aids on the hair.

Nonlimiting examples of styling agents suitable for use in the foamong compositions of the present invention include water-soluble materials such as polyalkylene glycols, polyethylene/polypropylene glycol copolymers, polyethylene/polypropylene diol copolymers, polyglycerins, and mixtures thereof, and/or their derivatives, and/or mixtures thereof, including the water-soluble polyalkylene glyceryl ethers which are also liquids or semisolids under ambient conditions. In this context, the term “water-soluble” refers to those styling materials that have a solubility in water at 25° C. of greater than 0.6%, preferably greater than 1.0%, more preferably greater than about 1.5% by weight.

Preferred styling agents suitable for use herein include those water-soluble polyalkylene glycols which conform to the formula:

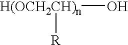

wherein R is selected from the group consisting of H, methyl, and mixtures thereof. When R is H, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist.

In the above structure, n has an average value of from 4 to about 35, preferably from about 5 to about 35, more preferably from about 5 to about 30, and even more preferably from about 5 to about 20.

Specific examples of preferred polyalkylene glycol polymers include polyethylene/polypropylene glycol copolymers (e.g., methoxy, ethoxy, propoxy, butoxy, and pentoxy, polyethylene/polypropylene glycols), triglycerin, hexaglycerin, PPG-4, PPG-6, PEG-5, PEG-6, PEG-8, PEG-12, PEG-14, PEG-18, PEG-20, PEG-32, and mixtures thereof. Most preferred are those polyalkylene glycols which have a number average molecular weight of from about 190 to about 1500, preferably from about 300 to about 1200, more preferably from about 400 to about 1000; and from about 5 to about 35, preferably from about 5 to about 30, more preferably from about 5 to about 20, repeating alkylene oxide radicals wherein each of the repeating alkylene oxide radicals has from 2 to 6 carbon atoms. Specific examples of the most preferred polyalkylene glycols include, but are not limited to, PPG-4 wherein R equals methyl and n has an average value of about 4; PEG-8 wherein R equals H and n has an average value of about 8 (PEG-8 is also known as Carbowax 400, which is available from Union Carbide); PEG-12 wherein R equals H and n has an average value of about 12 (PEG-12 is also known as Carbowax 600, which is available from Union Carbide); and PEG-20 wherein R equals H and n has an average value of about 20 (PEG-20 is also known as Carbowax 900, which is available from Union Carbide).

Optional Ingredients

### Thickening Agents

Commercially available carboxylic acid/carboxylate copolymers useful herein include: CTFA name Acrylates/C₁₀-₃₀ Alkyl Acrylate Crosspolymer having tradenames Pemulene TR-1™, Pemulene TR-2™, Carbopol 1342™, Carbopol 1382™, and Carbopol ETD 2020™, all available from B. F. Goodrich Company.

Neutralizing agents may be included to neutralize the carboxylic acid/carboxylate copolymers herein. Nonlimiting examples of such neutralizing agents include sodium hydroxide, potassium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

The concentration of the polymers typically ranges from about 0.01% to about 10%, preferably from about 0.05% to about 5%, more preferably from about 0.1% to about 2%, by weight of the composition.

### Plasticizer

The compositions hereof can optionally contain a plasticizer for the polymers. Any plasticizer suitable for use in hair care products or for topical application to the hair or skin can be used. A wide variety of plasticizers are known in the art. These include acetyl triethylcitrate, triethycitrate, glycerin, diisobutyl adipate, butyl stearate, and propylene glycol. Plasticizers are typically used at levels of from about 0.01% to about 10%, by weight of the composition, preferably from about 0.05% to about 3%, more preferably from about 0.05% to about 1%.

### Ionic Strength Modifier System

Optionally, the compositions of the present invention can contain an effective amount of a non-surface active ionic strength modifier system for reducing the viscosity of the hair spray composition. When used, the ionic strength modifiers will be present in the present compositions at a level of at least about 0.01%, by weight of the composition. The upper limit is dependent upon the maximum amount of the ionic strength modifiers that can be present in the particular compositions hereof such that the hair setting resin remains solubilized or dispersed. As will be understood by those skilled in the art, as the ionic strength of the composition is increased, the resin will eventually fall out of solution, or otherwise no longer remain solubilized or dispersed in the polar liquid carrier. The upper limit of the ionic strength modifier system level will vary depending upon the particular ionic strength modifiers, liquid vehicle, resin, and other ingredients present in the composition. Thus, for example, the maximum amount of the ionic strength modifiers that can be used will tend to be lower for compositions with liquid vehicles containing less water, compared to compositions with more water. Generally, the compositions will comprise about 4%, by weight, or less of the ionic strength modifiers, more generally about 2% or less, and typically about 1% or less. Preferably, the compositions hereof will comprise from about 0.01% to about 0.5%, more preferably from about 0.01% to about 0.1%, of the ionic strength modifier system.

The ionic strength modifier system comprises a mixture of monomeric cations and anions. The ions of the ionic strength modifier system hereof are non-surface active, i.e. they do not significantly reduce surface tension. For purposes hereof, non-surface active shall mean the ions, which at a 0.5% aqueous solution concentration, reduce surface tension by no more than 5.0 dynes/cm2. Generally, the ions of the ionic strength modifier system hereof will be characterized by having, at maximum, four or less carbon atoms per charge, preferably two or less carbon atoms, in any aliphatic chain or straight or branched chain organic heterochain.

The ionic strength modifier system comprises monomeric ions of the type which are products of acid-base reactions. Thus, basic and acidic ions OH⁻ and H⁺ do not constitute part of the ionic strength modifier system hereof, although they may be present in the composition. The ions hereof are incorporated into the composition in a form such that they can exist in the composition as free ions, i.e., in dissociated form. It is not necessary that all of the ions added exist in the composition as free ions, but must be at least partially soluble or dissociated in the composition. The ionic strength modifiers can be incorporated into the hair styling compositions, for example, by addition of soluble salts, or by addition of mixtures of acids and bases, or by a combination thereof. It is a necessary aspect of the invention that both anions and cations of the ionic strength modifier system be included in the composition.

Suitable cations for use include, for example, alkali metals, such as lithium, sodium, and potassium, and alkaline-earth metals, such as magnesium, calcium, and strontium. Preferred of the divalent cations is magnesium. Preferred monovalent metal ions are lithium, sodium, and potassium, particularly sodium and potassium. Suitable means of addition to the compositions hereof include, for example, addition as bases, e.g., hydroxides, sodium hydroxide and potassium hydroxide, and such as salts that are soluble in the liquid carrier, e.g. salts of monomeric anions such as those described below.

Other suitable cations include organic ions, such as quaternary ammonium ions and cationic amines, such as ammonium mono-, di-, and tri-ethanolamines, triethylamine, morpholine, aminomethylpropanol (AMP), aminoethylpropanediol, etc. Ammonium and the amines are preferably provided in the forms of salts, such as hydrochloride salts.

Monomeric anions that can be used include halogen ions, such as chloride, fluoride, bromide, and iodide, particularly chloride, sulfate, ethyl sulfate, methyl sulfate, cyclohexyl sulfamate, thiosulfate, toluene sulfonate, xylene sulfonate, citrate, nitrate, bicarbonate, adipate, succinate, saccharinate, benzoate, lactate, borate, isethionate, tartrate, and other monomeric anions that can exist in dissociated form in the hair styling composition. The anions can be added to the compositions hereof, for example, in the form of acids or salts which are at least partially soluble in the liquid vehicle, e.g., sodium or potassium salts of acetate, citrate, nitrate, chloride, sulfate, etc. Preferably, such salts are entirely soluble in the vehicle.

The use of ionic strength modifiers are especially useful in reduced volatile organic solvent compositions, most especially those utilizing silicone macromer-containing polymers.

### Nonpolar, Branched Chain Hydrocarbon

The compositions hereof may contain a volatile, nonpolar, branched chain hydrocarbon. The branched chain hydrocarbon solvent hereof may be present at a level of from about 0.1% to about 15%, preferably from about 0.5% to about 10%, more preferably from about 2% to about 8%, by weight of the composition.

The branched chain hydrocarbon solvent is characterized by a boiling point of at least about 105° C., preferably at least about 110° C., more preferably at least about 125° C., most preferably at least about 150° C. The boiling point is also generally about 260° C. or less, preferably about 200° C. or less. The hydrocarbon chosen should also be safe for topical application to the hair and skin.

The branched chain hydrocarbon solvents are selected from the group consisting of C₁₀-C₁₄ branched chain hydrocarbons, and mixtures thereof, preferably C₁₁-C₁₃ branched chain hydrocarbons, more preferably C₁₂ branched chain hydrocarbons. Saturated hydrocarbons are preferred, although it isn't necessarily intended to exclude unsaturated hydrocarbons.

Examples of suitable nonpolar solvents include isoparaffins of the above chain sizes. Isoparaffins are commercially available from Exxon Chemical Co. Examples include Isopar™ G (C₁₀-C₁₁ isoparaffins), Isopar™ H and K (C₁₁-C₁₂ isoparaffins), and Isopar™ L (C₁₁-C₁₃ isoparaffins). The most preferred nonpolar solvent are C₁₂ branched chain hydrocarbons, especially isododecane. Isododecane is commercially available from Preperse, Inc. (South Plainfield, N.J., USA) as Permethyl™ 99A.

The silicone macromer portion of the silicone grafted polymer is soluble in the nonpolar hydrocarbon solvent in the present compositions. This can be easily determined by verifying whether a silicone macromer of the same composition and molecular weight as that grafted to the silicone grafted polymer is soluble in the nonpolar hydrcarbon solvent. In general, the macromer should be soluble at 25° C. at a concentration of 0.1% by weight of the hydrocarbon solvent, preferably at 1%, more preferably at 5%, most preferably at 15%.

The nonpolar hydrocarbon solvent, however, is insoluble in the polar solvent of the composition. This is determined in the absence of the silicone grafted polymer, or other emulsifying agents, and can easily be verified by observing whether the polar and nonpolar solvents form separate phases after being mixed together.

Without intending to be necessarily limited by any particular theory, it is believed that the nonpolar hydrocarbon solvent solubilizes the silicone macromer portion of the silicone grafted polymer. This is believed to aid in obtaining a smoother polymer film upon drying. Since the hydrocarbon solvent is less volatile than the polar solvent phase, the hydrocarbon solvent maintains the silicone portions in solubilized form for a relatively long period as the composition dries, thus minimizing aggregation of the silicone portions and, therefore, allowing the polymer to dry as a smoother film.

### Hair Styling Compositions

The present invention encompasses a wide variety of hair styling compositions, including hair spray compositions, mousses, and hair setting tonics. In general, the compositions will be higher viscosity compositions that, preferably, are suitable for gel and mousse applications. Low viscosity hair spray are also contemplated, however.

### Method of Making

The compositions of present invention can be made conventional formulation and mixing techniques.

### Method of Use

The compositions of the present invention are used in conventional ways to provide the hair styling/holding benefits of the present invention. Such method generally involves application of an effective amount of the product to dry, slightly damp, or wet hair before and/or after the hair is arranged to a desired style. The composition is then dried or allowed to dry. By “effective amount” is meant an amount sufficient to provide the hair hold and style benefits desired considering the length and texture of the hair. In general, from about 0.5 g to about 50 g of product will be applied to the hair, depending upon the particular product formulation, dispenser type, length of hair, and type of hair style.

### EXAMPLES

The compositions illustrated in the following Examples illustrate specific embodiments of the hair styling compositions of the present invention, but are not intended to be limiting thereof. Other modifications can be undertaken by the skilled artisan without departing from the spirit and scope of this invention. These exemplified embodiments of the hair styling composition of the present invention provide styling and volumizing benefits.

The compositions illustrated in the following Examples are prepared by conventional formulation and mixing methods, an example of which is set forth hereinbelow. All exemplified amounts are listed as weight percents and exclude minor materials such as diluents, preservatives, color solutions, imagery ingredients, botanicals, and so forth, unless otherwise specified.

### Example 1

The following is a hair styling gel composition representative of the present invention.

| Ingredients | Weight % |
|---|---|
| Luviskol VA 37E (1) | 2.50 |
| Expancel DE091 (2) | 1.5 |
| Water | QS 100% |
| Carbomer 940 (3) | 0.50 |
| Panthenol | 0.05 |
| Polysorbate 80 | 0.20 |
| Perfume | 0.20 |

(1) available from BASF Corp.
(2) available from Akzo Nobel
(3) available from B. F. Goodrich Corp.

This product is prepared by dispersing the Luviskol VA 37E and Carbomer 940 in water. The mixture is stirred for approximately one half hour and the remaining components are added.

### Example 2

The following is a spray-on gel hair composition representative of the present invention.

| Ingredients | Weight % |
|---|---|
| Water | Q.S. to 100% |
| Ethanol | 15.00 |
| Panthenol | 0.05 |
| Perfume | 0.20 |
| Polyquaternium-11 (1) | 1.00 |
| Tospearl 240 (2) | 2.00 |

(1) available from ISP Corp.
(2) available from GE Silicones

This product is prepared by dissolving the Polyquaternium-11 in water and then adding the ethanol. The mixture is stirred for one half hour and the other components are mixed in.

### Example 3

The following is a hair styling mousse composition representative of the present invention.

| Ingredients | Weight % |
|---|---|
| Water | Q.S. to 100% |
| Lauramine Oxide | 0.20 |
| Panthenol | 0.05 |
| Perfume | 0.05 |
| Celquat-H100 (1) | 1.00 |
| Tospearl 240 | 1.5 |
| Isobutane | 7.00 |

(1) available from National Starch Corp.

This product is prepared by dissolving the Celquat-H100 polymer in water and mixing for one half hour. The other components (except isobutane) are added and mixed for an additional 10 minutes. Aluminum aerosol cans are then filled with 93 parts of this batch, affixed with a valve whcih is crimped into position, and lastly pressure filled with 7 parts Isobutane. This composition is useful for application to the hair to provide conditioning, styling and hold.

What is claimed is:

1. A hair styling composition comprising:
(a) from about 0.1% to about 15%, by weight, of an adhesive polymer having a weight average molecular weight of greater than about 20,000;
(b) from about 0.01% to about 20%, by weight, of particles; and
(c) an aqueous carrier.

2. The composition of claim 1 wherein at least about 0.1 weight percent of said particles are present.

3. The composition of claim 1 wherein at least about 0.5 weight percent of said particles are present.

4. The composition of claim 1 wherein said particles have a mean particle size of less than about 70 microns.

5. The composition of claim 1 wherein said particles have a mean particle size of less than about 60 microns.

6. The composition of claim 1 wherein said particles have a particle size range of about 2 to about 60 μm.

7. A method of treating hair by administering a safe and effective amount of the composition according to claim 1.
